(19) United States
(12) Patent Application Publication
(10) Pub. No.: US 2007/0048773 A1
(43) Pub. Date: Mar. 1, 2007

(54) Detection of polyphosphate using fluorescently labeled polyphosphate acceptor substrates

(75) Inventors: Linda G. Lee, Palo Alto, CA (US); Gerald Zon, San Carlos, CA (US); Karl O. Voss, Foster City, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation

(21) Appl. No.: 11/496,011

(22) Filed: Jul. 28, 2006

Related U.S. Application Data
(60) Provisional application No. 60/703,797, filed on Jul. 29, 2005.

(51) Int. Cl. C12Q 1/68; C12N 1/08

(52) U.S. Cl. 435/6; 435/270; 536/25.32

(57) ABSTRACT

Provided herein are methods and compositions for fluorogenic detection of a polyphosphate released from a nucleoside polyphosphate by the enzymatic action of a nucleic acid polymerase. The methods and compositions are based on the transfer of a free polyphosphate (or polyphosphate labeled with a fluorescence dye) to a polyphosphate acceptor molecule that is modified with a moiety that facilitates fluorogenic detection of a product that indicates the release of polyphosphate by the enzymatic action of a DNA or RNA polymerase. In one aspect, fluorogenic detection is facilitated by forming a fluorescent donor/acceptor pair on the polyphosphate acceptor substrate. The combination of the donor and acceptor pair provides a differentially detectable fluorescent product. In another aspect, fluorogenic detection is facilitated by releasing a fluorescent dye from the polyphosphate acceptor substrate when the free polyphosphate is transferred to the acceptor substrate. When the fluorescent dye is released form the acceptor substrate, it is dequenched, thereby providing for fluorescent detection.

### CROSS-REFERENCES TO RELATED APPLICATIONS

This application claims the benefit under 35 U.S.C. § 119(e) to U.S. Provisional Patent Application No. 60/703,797 filed Jul. 29, 2005, entitled DETECTION OF POLYPHOSPHATE USING FLUORESCENTLY LABELED POLYPHOSPHATE ACCEPTOR SUBSTRATES, the entire contents of which is incorporated herein by reference.

### FIELD

The present teachings are in the field of fluorescent detection of polyphosphates released by the enzymatic action of a nucleic acid polymerase.

### BACKGROUND

Sequencing techniques may be divided into two types based on the method used to identify nucleotides at a given position in the sequence. The first type involves first producing a set of labeled fragments of different sizes that correspond to each occurrence of a given nucleotide then separating the labeled fragments by size, typically using electrophoretic techniques. A second type of method, commonly referred to as “pyro-sequencing” is a real time method that relies on detecting pyrophosphate released when a nucleotide is incorporated into a de-novo strand by a DNA polymerase. The released pyrophosphate serves as a substrate for a sulfurylase, which acts in one of a series of coupled enzymatic reactions with other substrates culminating with production of a chemiluminescent product by the action of luciferase. One of the coupled enzymes included in the reaction mixture is a nucleotide degrading enzyme (i.e., apyrase) that degrades excess amounts of the added nucleotide and regenerates the initial substrate used for binding to the released pyrophosphate. Pyro-sequencing requires at least four enzymes (DNA polymerase, sulfurylase, luciferase, and apyrase) and is based on stepwise addition of nucleotide triphosphates to a reaction mixture with chemiluminescent detection.

There is a need in the art for better methods of stepwise sequencing that are more cost effective and that provide more sensitive detection than chemiluminescent methods used in conventional pyro-sequencing methods. The present invention addresses this and other needs.

### SUMMARY

Provided herein are methods and compositions for analyzing, identifying, or sequencing nucleotides in a sample nucleic acid by fluorogenic detection of a polyphosphate released from a nucleoside polyphosphate. The methods and compositions are based on recognizing that a polyphosphate (or polyphosphate labeled with a fluorescent dye) can be transferred to a polyphosphate acceptor substrate, exemplified herein by adenosine 5′ phosphosulfate (APS), that is modified with a moiety that facilitates fluorogenic detection of a product formed when the released polyphosphate is transferred to the polyphosphate acceptor substrate.

In one aspect, fluorogenic detection is facilitated by forming a fluorescent donor/acceptor pair on the polyphosphate acceptor substrate. This aspect includes reacting a sample nucleic acid comprising a targeted nucleotide with a nucleotide polymerase, a nucleic acid primer, and a nucleoside polyphosphate molecule that complements the targeted nucleotide and is labeled with a first dye. After a time sufficient to release a first dye labeled polyphosphate molecule from the nucleoside polyphosphate molecule, the first dye-labeled polyphosphate is reacted with a polyphosphate acceptor substrate labeled with a second dye in the presence of a polyphosphate transfer enzyme to form a polyphosphate acceptor substrate labeled with both the first dye and second dye. One of the first dye and second dye is a fluorescence donor dye and the other dye is a fluorescence acceptor dye. When excitation radiation within the absorption spectrum of the donor dye, the acceptor dye emits radiation, thereby identifying the presence of the targeted nucleotide. In additional embodiments, the process can be repeated using different nucleoside polyphosphates and the identity of the targeted nucleotide can be identified. The method can be repeated for successive nucleosides to sequence the sample nucleic acid.

In another aspect, fluorogenic detection is facilitated by releasing a fluorescent dye from the polyphosphate acceptor substrate. When a polyphosphate is released from a nucleoside polyphosphate by the action of a polymerase, it is transferred to the acceptor substrate and the transfer releases the fluorescent dye. When the fluorescent dye is released from the acceptor substrate, it is dequenched, thereby providing for fluorescent detection. Embodiments within this aspect do not depend on use of labeled nucleoside polyphosphates. In certain embodiments, the acceptor substrate is labeled with both the fluorescent dye and a quenching moiety that quenches the fluorescent dye. An exemplary embodiment using this method includes contacting a sample nucleic acid comprising a targeted nucleotide, a nucleic acid polymerase and a first nucleoside polyphosphate, with a polyphosphate transfer enzyme and polyphosphate acceptor substrate. The acceptor substrate is dually labeled with a first dye, which is a fluorescent dye, and with a second dye, which is a corresponding fluorescence quenching dye. When the polyphosphate is transferred to the polyphosphate acceptor substrate by the action of the polyphosphate transfer enzyme, the fluorescent dye is released from the polyphosphate acceptor substrate and thereby dequenched. Detecting the presence of the dequenched fluorescent dye indicates that the polymerase has released the polyphosphate from the nucleoside polyphosphate and that the transfer enzyme has transferred the released polyphosphate to the acceptor substrate.

The methods and compositions are useful for detecting polyphosphate in a sample, for analyzing a sample nucleotide, for sequencing a sample nucleotide, or for any purpose where detection of a polyphosphate released from a nucleoside polyphosphate by a polymerase is desirable. The methods provided herein can be practiced with any polymerase, including both DNA and RNA polymerases.

### BRIEF DESCRIPTION OF THE DRAWINGS

Aspects of the invention can be more fully understood with respect to the following drawings.

FIG. 1 depicts an exemplary embodiment of one aspect of the present teachings, which is a method for detecting a polyphosphate by forming a fluorescent donor/acceptor pair on a polyphosphate acceptor substrate.

FIG. 2 depicts an exemplary embodiment of another aspect of the present teachings, which is a method for detecting a polyphosphate by release of a fluorophore from a polyphosphate acceptor substrate having a quenching moiety attached thereto.

### DESCRIPTION OF EXEMPLARY EMBODIMENTS

I. Detection of Released Polyphosphate by Forming Acceptor Substrate with Donor/Acceptor Dyes

In one aspect, methods are provided for detecting release of a polyphosphate by the enzymatic action of a polymerase based on energy transfer between donor and acceptor dyes on a polyphosphate acceptor substrate. This aspect includes contacting a sample nucleic acid comprising the targeted nucleotide with a nucleotide polymerase, a nucleic acid primer, and a nucleoside polyphosphate molecule that complements the targeted nucleotide and is labeled with a first dye. After a time sufficient to release a first dye labeled polyphosphate molecule, the first dye-labeled polyphosphate is reacted with a polyphosphate acceptor substrate labeled with a second dye in the presence of polyphosphate transfer enzyme to form a polyphosphate acceptor substrate labeled with both the first dye and second dye. One of the first dye and second dye is a fluorescence donor dye and the other dye is a fluorescence acceptor dye. When excitation radiation within the absorption spectrum of the donor dye, the acceptor dye emits radiation, thereby identifying the presence of the targeted nucleotide. In additional embodiments, the process can be repeated using different nucleoside polyphosphates and the identity of the targeted nucleotide can be identified. The method can be repeated for successive targeted nucleosides to sequence the sample nucleic acid.

As used herein, a “fluorescent acceptor dye corresponding to the donor dye” means the donor dye transfers electrons, photons or other form of energy to the acceptor dye in a manner that activates the acceptor dye to emit photons or to be put into a state where photons can be emitted upon excitation of the donor or acceptor dye with light of a suitable energy. In certain embodiments, the donor/acceptor combination depends on spectral overlap between the donor and acceptor dye and functions at distance (i.e., by fluorescence resonance energy transfer, FRET). In other embodiments, the donor and acceptor interact between molecular orbitals and require contact between the donor and acceptor to transfer electrons from the donor to the acceptor. In various embodiments, excitation of the donor dye by light of suitable wavelength is required to transfer electrons or photons between the donor and acceptor dyes. All such donor/acceptor mechanisms are included in the meaning of “fluorescent acceptor energy corresponding to a fluorescent donor energy” and grammatical variations of the same.

The type of nucleoside polyphosphates useful in this aspect can be any nucleoside polyphosphate (or analogue thereof that can be used as a substrate by a nucleic acid polymerase) having 3 or more phosphate residues attached to the 5′ position of the sugar moiety of the nucleoside. The terminal phosphate group of the nucleoside polyphosphate is labeled with a fluorescent dye with a donor transfer energy and is differentially detectable when combined with the polyphosphate acceptor molecule labeled with the second dye having a corresponding acceptor energy. As used in this context, “differentially detectable” means there is some selected excitation and/or emission wavelength where the polyphosphate acceptor molecule labeled with both the fluorescent donor dye and the second acceptor dye, can be distinguished from the donor dye labeled on the terminal phosphate of the nucleoside polyphosphate, from the acceptor substrate labeled with the acceptor dye alone, and from a polyphosphate labeled with the first fluorescent dye alone.

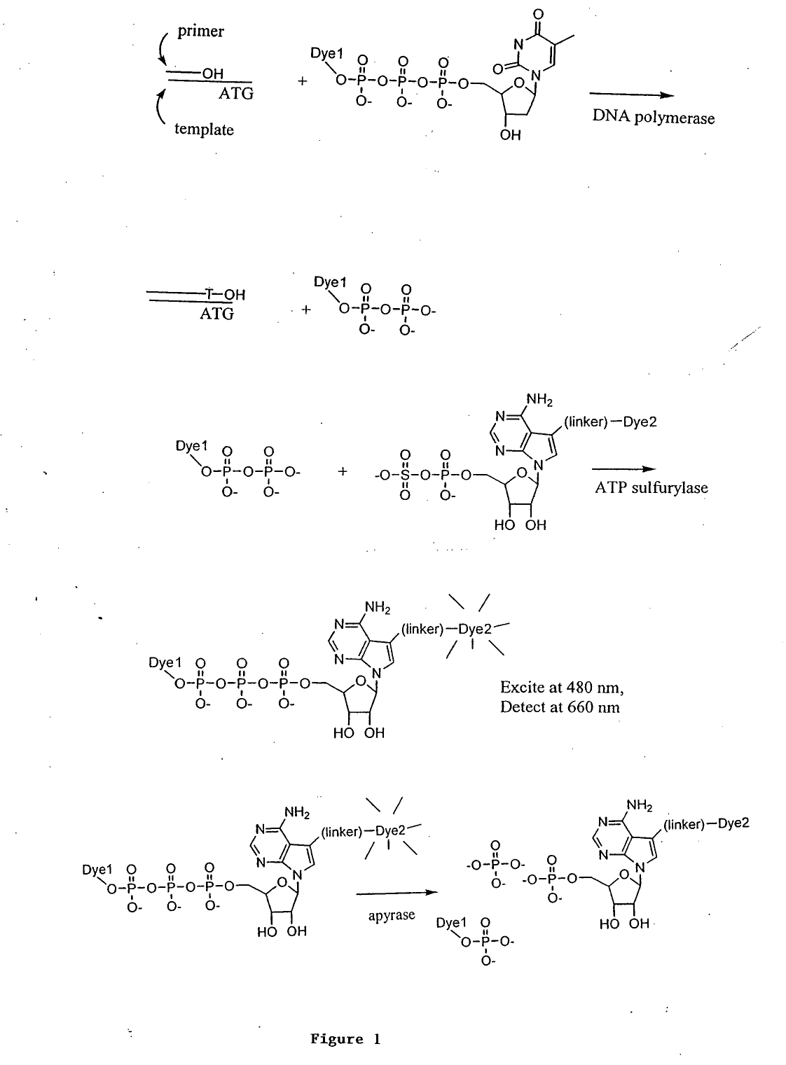

FIG. 1 illustrates an exemplary embodiment of this aspect of the disclosure. A nucleoside triphosphate is labeled at the terminal phosphate with a first dye, generically depicted as “dye 1.” Dye 1 can be any dye, and in some embodiments is selected to be a “caged dye,” meaning that the dye is not fluorescent when linked to a one or more phosphate groups alone, or when the phosphate group(s) are further attached to the nucleoside. In the presence of a single stranded sample nucleic acid (template), and a polymerase, if the nucleoside triphosphate is complementary to the next base on the template, the nucleoside monophosphate is incorporated into the next adjacent position on the primer with release of a polyphosphate (depicted as pyrophosphate) labeled with the donor dye.

PCT publication WO 2004/072238, which is incorporated herein by reference, demonstrated that with appropriate reaction conditions (i.e., in the presence of Mn⁺² ions), dye labeled nucleoside polyphosphates containing three or more phosphate groups can be used as substrates for a variety of DNA polymerases. It was previously known that RNA polymerases, which are more promiscuous than DNA polymerases with respect to nucleotide utilization, are capable of using terminal phosphate labeled nucleosides. Hence, the methods provided herein can be conducted with a RNA polymerase or a DNA polymerase. The reaction depicted in FIG. 1, is illustrated with a DNA polymerase, in which case a single stranded sample nucleotide is used as the template, and a primer complementary to a portion of the sample nucleotide is hybridized to the primer. If an RNA polymerase is used, the sample nucleotide is typically double stranded, and contains either an RNA polymerase promoter sequence or site for end initiation by the RNA polymerase.

In the presence of a polyphosphate transfer enzyme, or a mutant transfer enzyme, and corresponding polyphosphate acceptor substrate, the polyphosphate labeled with the donor dye 1 is transferred to the acceptor substrate. As depicted in FIG. 1, the polyphosphate transfer enzyme is ATP sulfurylase, or a mutant thereof, the polyphosphate acceptor substrate is adenosine 5′ phosphosulfate (APS), and the enzymatic activity of the sulfurylase transfers the polyphosphate labeled with the donor dye to the alpha phosphate group of the APS. As used herein, the term “ATP sulfurase” refers to wild-type ATP sulfurase and mutants thereof. The APS is labeled on the base with a second dye, depicted as “dye 2.” Dye 2 is selected to have a donor acceptor energy that corresponds with the donor energy of dye 1. When the donor and acceptor dyes are linked to the same substrate and illuminated with a wavelength suitable to excite the donor dye, the energy of the donor dye is transferred to the acceptor dye and fluorescence is detected at an appropriate wavelength. Because light emitted at the selected wavelength is differentially detectable from light emitted from either the nucleoside polyphosphate labeled with the donor dye, the labeled polyphosphate or the polyphosphate acceptor substrate labeled with the acceptor dye, the detection of fluorescence indicates that the dye labeled polyphosphate has been released by the enzymatic action of the polymerase. The donor and acceptor dye can be interchangeably on the nucleoside polyphosphate or polyphosphate acceptor substrate.

In various embodiments, the reaction mixture can further include a substrate degrading enzyme, or combination of enzymes, that degrades the dually labeled polyphosphate acceptor substrate. The degrading enzyme or combination of enzymes, releases the donor dye from the polyphosphate acceptor substrate, removing it from proximity to the acceptor dye, thereby causing the detected fluorescence to decline. In various embodiments the substrate degrading enzyme or combination thereof can also be selected to degrade the nucleoside polyphosphate. As exemplified in FIG. 1, apyrase is used as the substrate degrading enzyme. As used herein, the term “apyrase” refers to wild-type apyrases and mutants thereof. Advantageously, a mutant apyrase can degrade both the nucleoside polyphosphate and the dually labeled polyphosphate acceptor substrate.

In other embodiments, other enzymes or enzyme combinations that provide these degrading activities can be used. One example of another class of enzymes that can provide this degrading activity is phosphodiesterases. Phosphodiesterases cleave phosphoester linkages on either side of a phosphate linked to two or more other phosphates, but do not cleave between a phosphate linked to other moieties. Another example substrate degrading enzyme is pig pancreas nucleoside triphosphate diphosphohydrolase (Le Bel et al., 1980, J. Biol. Chem., 255, 1227-1233). In general, any enzyme that is capable of degrading the polyphosphate acceptor substrate alone, or in addition to degrading the nucleoside polyphosphate may be used. Because APS is a modified nucleoside, any enzyme capable of degrading nucleosides is acceptable, including enzymes that degrade the base, the terminal phosphates, or the sugar moiety.

As will be described in more detail hereafter, the methods provided herein can be practiced in a variety of embodiments. In certain embodiments, the methods are performed with transfer of reagents from a first reaction mixture containing a complex of the polymerase and the sample nucleotide immobilized on a substrate, to a second reaction mixture containing the polyphosphate transfer enzyme and acceptor substrate. Substrate degrading enzymes may optionally be included in the second reaction mixture.

In other embodiments, the reaction mixture simultaneously contains the sample nucleotide, the polymerase, the labeled nucleoside polyphosphate, the polyphosphate transfer enzyme and the substrate degrading enzyme. In such embodiments, to assure detection of the dually labeled polyphosphate acceptor substrate, the substrate degrading enzyme(s) is used in an amount, or under reaction conditions selected to slowly degrade the dually labeled acceptor substrate. In this context, “slowly degrade” means that for a given concentration of polyphosphate acceptor substrate used in the reaction mixture, the substrate degrading enzyme will not degrade the dually labeled polyphosphate acceptor substrate for at least a period of time sufficient to first detect the presence of the dually labeled acceptor substrate. The amount of substrate degrading enzyme used in the reaction will depend on several parameters, including for example, the relative rate of incorporation of the nucleosides in a growing polynucleotide chain by the polymerase, the amount of sample nucleotide in the reaction mixture and the relative lumetic properties of the substrate degrading enzyme and the polyphosphate transfer enzyme. The amount of substrate degrading enzyme used in the reaction mixture thus depends on several factors. Generally speaking, the substrate degrading enzyme is selected to have kinetic characteristics relative to the polyphosphate transfer enzyme such that the labeled polyphosphate is first efficiently transferred to the polyphosphate acceptor substrate and remains for a sufficient period of time to detect the dually labeled substrate. Thus, for example, if the Km of the polyphosphate transfer enzyme is relatively low in comparison to the Km of the substrate degrading enzyme, then a lower amount of the substrate degrading enzyme would be used in comparison of the amount of polyphosphate transfer enzyme.

In embodiments that use sulfurylase as the polyphosphate transfer enzyme, APS as the polyphosphate acceptor substrate, and apyrase as one of the substrate degrading enzymes, sulfate can optionally be included in the reaction mixture, in which case the sulfurylase will transfer the sulfate group to the phosphate group of the adenosine monophosphate moiety formed by activity of the apyrase, thereby regenerating the APS. In other optional embodiments, the APS can be present in sufficient excess so that several cycles of nucleoside polyphosphate addition can occur without depleting the APS in the reaction mixture below the level need to receive additional labeled polyphosphates added in subsequent intervals.

If no fluorescent emission is detected in the presence of the first nucleoside polyphosphate, a second terminal phosphate labeled nucleoside polyphosphate different from the first, but also labeled with a dye having the donor transfer energy can be subsequently added to determine if light is emitted. The process can be repeated with a third and fourth terminal labeled nucleoside polyphosphates until an emission is detected. By knowing the identity of the labeled nucleoside polyphosphate added at any given interval, the identity of the complementary base on the nucleic acid template can be determined.

The dye on the second nucleoside polyphosphates may be same or different from the dye on the first nucleoside polyphosphates so long as the dye functions as a donor dye for the acceptor dye on the polyphosphate acceptor substrate. Numerous donor/acceptor dye pairs can be used in the methods provided herein, where the donor dye is initially linked to the terminal phosphate of a nucleoside polyphosphates and the acceptor dye is linked to the polyphosphate acceptor substrate. Non-limiting examples of suitable of donor/acceptor pairs include, but are not limited to, fluorescein/Symjaz 660, fluorescein/rhodamine, rhodamine/fluorescein, xanthene/cyanine, xanthene/pthalocyanine, xanthene/rhodamine and xanthene/squaraine. These classes of donor/acceptor pairs also include any of a number of derivatives of the core dye species, which can contain any of a variety of functional groups or modifications that may alter particular properties of the core species.

II. Detection of Polyphosphate by Release of a Fluorescent Dye from a Polyphosphate Acceptor Substrate

In another aspect, there is provided a method detecting the presence of a polyphosphate in a sample that includes the acts of contacting the sample with a polyphosphate transfer enzyme and a polyphosphate acceptor substrate labeled with a fluorescent dye at an acceptor site where the fluorescent dye is quenched when linked to the acceptor substrate. Fluorescence from the fluorescent dye is detected when the polyphosphate is transferred to the acceptor site on the acceptor substrate and releases the fluorescent dye from the acceptor substrate by enzymatic action of the polyphosphate transfer enzyme. Embodiments of this aspect are useful for detecting the presence of a polyphosphate in any type of sample.

In certain embodiments, the method is used for determining if a polyphosphate is released from a nucleoside polyphosphate by the enzymatic action of a polymerase based on releasing a fluorescent dye from a polyphosphate acceptor substrate when the released polyphosphate is transferred to the acceptor substrate. Embodiments that use this aspect do not depend on use of labeled nucleoside polyphosphates. This method comprises reacting a sample nucleic acid comprising a targeted nucleotide, the nucleic acid polymerase and a first nucleoside polyphosphate, with a polyphosphate transfer enzyme and polyphosphate acceptor substrate. The polyphosphate acceptor substrate is labeled with a fluorescent dye that is differentially detectable when released from the polyphosphate acceptor substrate in comparison to when attached to the polyphosphate acceptor substrate. Release of the fluorescent dye from the polyphosphate acceptor substrate is accomplished by the activity of the polyphosphate transfer enzyme.

In certain embodiments, the acceptor substrate is dually labeled with a first dye, which is a fluorescent dye, and with a second dye, which is a corresponding fluorescence quenching dye. As used herein, “a corresponding fluorescence quenching dye” is a second dye that is capable of quenching the fluorescence of the first dye when both are present on the polyphosphate acceptor substrate. When the polyphosphate is transferred to the polyphosphate acceptor substrate by the action of the polyphosphate transfer enzyme, the fluorescent dye is released from the polyphosphate acceptor substrate and thereby dequenched. Detecting the presence of the dequenched fluorescent dye indicates that the polymerase has released the polyphosphate from the nucleoside polyphosphate and that the transfer enzyme has transferred the released polyphosphate to the acceptor substrate.

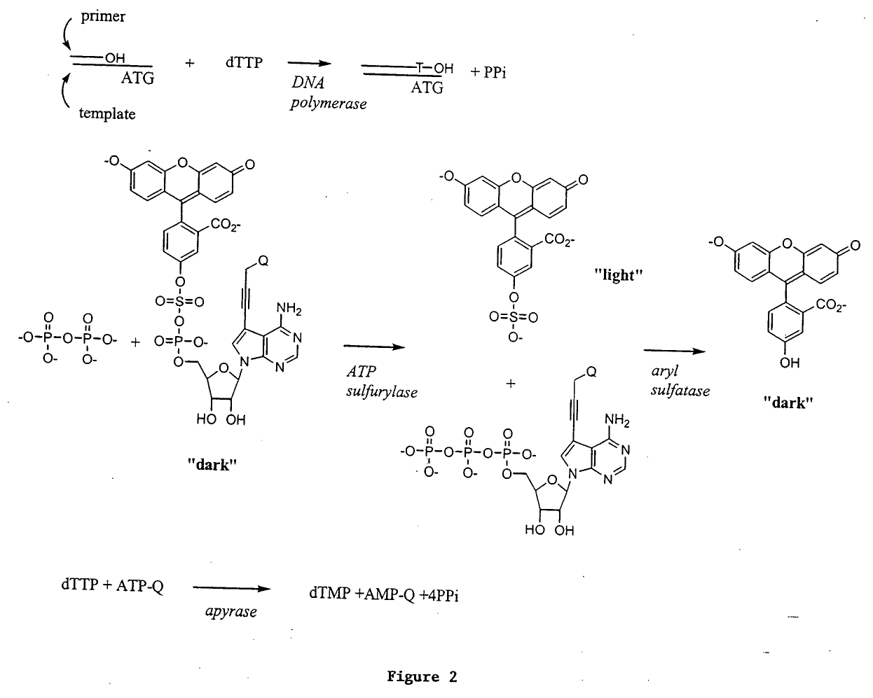

FIG. 2 illustrates and exemplary embodiment of this aspect of the methods and compositions provided herein. In the example depicted in FIG. 2, the polyphosphate transfer enzyme is ATP sulfurylase and the polyphosphate acceptor substrate is APS. The APS is labeled at the terminal sulfate group with a xanthene donor dye and labeled on the base with a quenching dye. The presence of the quenching dye suppresses fluorescent emission form the xanthene donor dye. When the polyphosphate (i.e., pyrophosphate) is released from the nucleoside triphosphate, the pyrophosphate is transferred by the sulfurylase to the alpha phosphate of the APS releasing a sulfated xanthene dye and forming ATP labeled only with the quenching dye. The fluorescence of the sulfated xanthene dye is detected when it is removed from physical proximity to the quenching dye.

Optionally, the reaction mixture can further include a substrate degrading enzyme or combination of enzymes that degrades the released fluorescent dye and/or the nucleoside polyphosphate and/or the polyphosphate acceptor substrate. In certain embodiments, the substrate degrading enzyme degrades the fluorescence emitted from the released fluorescence dye. In other embodiments, the substrate degrading enzyme activates the emission of fluorescence from the released fluorescent dye. In the example embodiment depicted in FIG. 2, the sulfated fluorescent dye released from the polyphosphate acceptor substrate has greater fluorescence when the sulfate is attached to the xanthene dye and aryl sulfatase is used to degrade the sulfated xanthene dye, releasing the sulfate group, which reduces or eliminates the fluorescence of the xanthene dye. In other embodiments, where the fluorescent dye is a “caged” dye, the released dye has greater fluorescence when the sulfate is removed from dye by the action of the sulfatase. In such cases, the released fluorescent dye must be removed from the reaction mixture or otherwise degraded to lower the detected fluorescence prior to adding a second nucleoside polyphosphate to the reaction mixture.

In either case, the substrate degrading enzymes can further include a nucleotide degrading enzyme such as apyrase that degrades the first nucleoside polyphosphate present in the reaction mixture as well as the ATP formed by the action of sulfurylase. Other enzymes that will degrade the nucleoside polyphosphate and the acceptor substrate include, but are not limited to, phosphodiesterases and phosphatases.

In certain embodiments, the fluorescent dye is an organic dye derivatized for attachment to the acceptor substrate at the site of transfer of the polyphosphate. The fluorescent dye can be attached directly to the acceptor substrate, or via a linker. For example, as depicted in FIG. 2, the xanthene dye is attached to the sulfate group of APS. The quencher dyes are typically also organic dyes, which may or may not be fluorescent. In some embodiments, the quenching operates by a fluorescent resonance energy transfer mechanism, akin to the donor/acceptor mechanism, except that the energy transferred to the quenching dye quenches the fluorescent of the fluorescent dye rather than inducing fluorescence of an acceptor dye. Therefore, in some embodiments, the released fluorescent dye and the quencher dye attached to the polyphosphate acceptor substrate can both be fluorescent. In such cases, all that is required is that the released fluorescent dye is differentially detectable when released from the quenching dye attached to the polyphosphate acceptor substrate.

In other embodiments, the fluorescent dye and quenching dye function by an electron transfer mechanism. For example, a non-fluorescent quenching dye such as DABCYL or dinitrophenyl absorbs energy from the excited fluorescent dye, but does not release the energy radiatively. These quenching dyes can be referred to as chromogenic dyes. Many fluorescent/quenching dye combinations can be used in the methods provided herein. There is a great deal of practical guidance available in the literature for providing an exhaustive list of fluorescent and chromogenic molecules and their relevant optical properties. The following guides are incorporated herein by reference, to the extent they teach the relevant fluorescent and quenching properties of dyes: Berlman, Handbook of Fluorescence Spectra of Aromatic Molecules, 2nd Edition (Academic Press, New York, 1971); Griffiths, Colour and Constitution of Organic Molecules (Academic Press, New York, 1976); Bishop, Ed., Indicators (Pergamon Press, Oxford, 1972); Haugland, Handbook of Fluorescent Probes and Research Chemicals (Molecular Probes, Eugene, 1992) Pringsheim, Fluorescence and Phosphorescence (Interscience Publishers, New York, 1949). Also available in the technical literature are guides for derivatizing various fluorescent and quencher dyes for covalent attachment via common reactive groups that can be added to a the polyphosphate substrate acceptor, especially when the acceptor substrate is itself a nucleoside. Suitable guidance can be found, for example, in Haugland (supra); Ullman et al., U.S. Pat. No. 3,996,345; and Khanna et al., U.S. Pat. No. 4,351,760, each incorporated herein by reference.

Suitable fluorescent dyes and quenching dyes operating on the principle of fluorescence energy transfer include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3 5 disulfonate; N-(4-anilino-1-naphthyl)maleimide; anthranilamide; BODIPY; Brilliant Yellow; coumarin and derivatives: coumarin, 7-amino-4-methylcoumarin (AMC, Coumarin 120), 7-amino-4- trifluoromethylcouluarin (Coumaran 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)4-methylcoumarin; diethylenetriamine pentaacetate; 4,4′-diisothiocyanatodihydro-stilbene-2,-2′-disulfonic acid; 4,4′- diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives: eosin, eosin isothocyanate, erythrosin and derivatives: erythrosin B, erythrosin, isothiocyanate; ethdium; fluorescein and derivatives: 5-carboxyfluorescein (FAM), 5-(4,6-dichlorotriazin-2-yl)aminofluorescein (DTAF), 2′,7′-dimethoxy4′5′-dichloro-6-carboxyfluorescein (JOE), fluorescein, fluorescein isothiocyanate, QFITC, (XRITC); fluorescamine; IR144; IR1446; Malachite Green isothocyanate; 4-methylumbelliferoneortho cresolphthalein; nitrotyrosine; pararosaniline; Phenol Red; B-phycoerythnn; o-phthaldialdehyde; pyrene and derivatives: pyrene, pyrene butyrate, succinimidyl 1-pyrene; butyrate quantum dots; Reactive Red 4 (Cibacron.TM. Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; terbium chelate derivatives; Cy 3; Cy 5; Cy 5.5; Cy 7; IRD 700; IRD 800; La Jolla Blue; phthalo cyanine; and naphthalo cyanine.

Typical fluorescent dyes that can be quenched by a suitable quenching dye include, but are not limited to, xanthene dyes, such as fluorescein, in combination with rhodamine dyes. Many suitable forms of these compounds are widely available commercially with substituents on their phenyl moieties that can be used as the site for bonding or as the bonding functionality for attachment to a functional group on the polyphosphate acceptor substrate.

Another group of suitable fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-toluidinyl-6-naphthalene sulfonate. Other dyes include 3-phenyl-7-isocyanatocoumarin, acridines, such as 9-isothocyanatoacridin-e and acridine orange; N-(p-(2-benzoxazolyl)phenyl)maleimide; benzoxadiazoles, stilbenes, pyrenes, and the like. Typically, the fluorophore/quencher pair are selected from fluorescein and rhodamine dyes. These dyes and appropriate linking methodologies for attachment to nucleotides are described in many references. Of these, Khanna et al. (cited above); Marshall, Histochemical J., 7:299-303 (1975); Menchen et al., U.S. Pat. No. 5,188,934; Menchen et al., European Patent Application 873 10256.0; and U.S. Pat. No. 5,366,860, to Bergot et al, each incorporated herein by reference, are useful resources in this regard.

In other typical embodiments, the quencher 4-(4′-dimethylaminophenylazo)-benzoic acid (DABCYL) is used. DABCYL quenches fluorescence from a wide variety of dyes emitting between 475 nm and 805 nm, with measured efficiencies ranging from 90 to 99.9% (see, S. Tyagi et al., Nat. Biotechnol. 16, 49 (1998); and G. T. Wang et al., Tetrahedron Lett. 31, 6493 (1990)). Without being bound by any particular theory, it is believed that the quenching mechanism of DABCYL probably involves electron transfer, rather than fluorescence resonance energy transfer, because it is wave length independent. In other typical embodiments, the quenchers dinitrophenyl (DNP) or trinitrophenyl (TNP) are used.

In other embodiments, fluorescent dyes, donors, acceptors, and/or quenchers can be covalently attached to a molecule by a linker. The linker can be any covalent molecule known in the art that can be used to covalently link two molecules together. Examples of linkers, include, but are not limited to, straight chains with 1, 2, 3, 4, 5, 6, 7, 8, 9, 10, 15, 20, 25, 30 carbon atoms. In such exemplary linkers, heteroatoms such as nitrogen, sulfur, and oxygen can be substituted for the carbon atoms. In addition, side chains can extend from the straight chain portion of the linker. The linker can also include alkyl, alkenyl, alkynyl, and aryl groups.

III. Assays With Polymerases

As mentioned before, the methods provided herein may be used with any DNA polymerase or RNA polymerase. It is desirable to use polymerases that have a relatively high processivity so that the complex between the DNA polymerase and the sample nucleotide remains bound through sequential steps in the methods. Processivity is typically measured by determining the average number of nucleotides incorporated by the polymerase in a given time period. A suitably processive polymerase incorporates at least 20 nucleotides per second, at least 200 nucleotides per second, at least 2000 nucleotides per second or at least 20,000 nucleotides per second. Suitable DNA polymerase include, but are not limited to, DNA polymerase I, the large fragment of DNA polymerase I (Klenow), reverse transcriptase, T7 DNA polymerase, Sequenase Ver. 2.0 (USB U.S.A.), Thermus aquaticus DNA polymerase (Taq polymerase), mitochondnal polymerase gamma, Phi-29 DNA polymerase, Pyrococcusfuriosus DNA polymerase (Pfu polymerase) as well as any of a variety of mutated versions of the same. Typically, it is desirable to use a DNA polymerases that is mutated to remove the 3′ exonuclease activity. It is known that many polymerases have a proof-reading or error checking ability and that 3′ ends available for chain extension are sometimes digested by one or more nucleotides. If such digestion occurs in the methods provided herein, the level of background noise may increase.

In embodiments that use a DNA polymerase, the reaction mixture contains a suitable primer that binds the sample nucleic acid to serves as the polymerase initiation site. Any suitable primer may be used. For example, an oligonucleotide containing the complement of a universal primer may be ligated to the end of a double stranded sample nucleic acid, and then the double stranded nucleic acid separated into single strands. The single stranded molecules are then hybridized to the universal primer. In certain embodiments, as in the case of genotyping mentioned below, the primer may be selected to bind to known sequences on the sample nucleic acid adjacent to a nucleotide polymorphism to be analyzed. In optional embodiments, the hybridizing portions of the primer may include a non-exonuclease digestible bond such as a phosphothioate bond to protect the primer from the 3′ exonuclease activity of the polymerase.

Alternatively, a primer with a phosphorylated 5′-end, containing a loop and annealing back on itself and the 3′-end of the single stranded template can be used. If the 3′-end of the template has the sequence region denoted T (template), the primer has the following sequence starting from the 5′-end; P-L-P′-T′, where P is primer specific (5 to 30 nucleotides), L is loop (preferably 4 to 10 nucleotides), P′ is complementary to P (preferably 5 and 30 nucleotides) and T′ is complementary to the template sequence in the 3′-end (T) (at least 4 nucleotides). This primer can then be ligated to the single stranded template. Alternatively, such a loop primer can be attached to the sample nucleic acid according to the method taught in W093/23563 incorporated herein by reference, which uses PCR to introduce loop structures that provide a permanently attached 3′ primer at the terminal end of the sample nucleic acid sequence. In optional embodiments, the hybridizing portions of the primer may include a non-exonuclease , digestible bond such as a phosphothioate bond to protect the primer from the 3′ exonuclease activity of the polymerase.

In other embodiments, the methods provided herein use RNA polymerases. Any RNA polymerase is suitable, however, RNA polymerases having well defined and specific promoter sequences are desirable. Highly processive single subunit RNA polymerases with well defined and specific promoter sequences are widely available from a variety of commercial sources. In typical embodiments, the RNA polymerase is a single subunit RNA polymerase encoded by bacterial phages such as the T7, T3 and SP6 RNA polymerases, each of which have well defined promoter sequences that can be obtained in cloning vectors or synthesized as oligonucleotides and then ligated to the end of a double stranded sample nucleic acid. As an alternative to ligating a promoter sequence, the double stranded sample nucleic acid may be used without being ligated to the promoter sequence, in which case RNA polymerase will initiate transcription at the end of the sample nucleic acid. In such cases, the sample nucleic acid can be bound to a substrate at one end or otherwise blocked at one end, so that end initiation will only occur at the free end.

Whether using a DNA polymerase or RNA polymerase, the sample nucleic acid may be amplified, and any method of amplification may be used, for example in vitro by PCR or Self Sustained Sequence Replication (3SR) or in vivo using a vector and, if desired, in vitro and in vivo amplification may be used in combination.

Whatever method used, the procedures provided herein may be modified so that the sample nucleic acid becomes immobilized or is provided with means for attachment to a solid support. For example, a PCR primer may be immobilized or be provided with means for attachment to a solid support. Immobilization of the amplified DNA may take place as part of PCR amplification itself, as where one or more primers are attached to a support, or alternatively, one or more of the PCR primers may carry a functional group permitting subsequent immobilization, e.g. a biotin or thiol group. Immobilization via the 5′ end of a primer allows the strand of DNA emanating from that primer to be attached to a solid support with its 3′ end remote from the support for use as the extension primer for subsequent hybridization to the sample nucleic acid and extension by the DNA polymerase.

The solid support may conveniently take the form of microtiter wells, which are advantageously in the conventional 8×12 format, or dipsticks which may be made of polystyrene activated to bind the primer DNA (K Almer, Doctoral Theses, Royal Institute of Technology, Stockholm, Sweden, 1988). However, any solid support may conveniently be used including any of the vast number described in the art, i.e. for separation/immobilization reactions or solid phase assays. Thus, the support may also comprise particles, fibers or capillaries made, for example, of agarose, cellulose, alginate, Teflon or polystyrene. Magnetic particles e.g. the superparamagnetic beads produced by Dynal AS (Oslo, Norway) also may be used as a support.

The solid support may carry functional groups such as hydroxyl, carboxyl, aldehyde or amino groups, or other moieties such as avidin or streptavidin, for the attachment of primers. These may in general be provided by treating the support to provide a surface coating of a polymer carrying one of such functional groups, i.e. polyurethane together with a polyglycol to provide hydroxyl groups, or a cellulose derivative to provide hydroxyl groups, a polymer or copolymer of acrylic acid or methacrylic acid to provide carboxyl groups or an aminoalkylated polymer to provide amino groups. U.S. Pat. No. 4,654,267, incorporated herein by reference, describes the introduction of many such surface coatings.

An alternative format for the analysis is to use an array format wherein samples are distributed over a surface, for example a microfabricated chip, and thereby an ordered set of samples may be immobilized in a 2-dimensional format. Many samples can thereby be analyzed in parallel.

In certain embodiments, the methods and compositions provided herein are used for single molecule sequencing. Single molecule sequencing differs from bulk sequencing in that only a single sample nucleotide molecule analyzed. Single molecule sequencing does not require synchronization of the polymerization reaction for a plurality of sample nucleic acids because the detection events detect incorporation of nucleotides into a single growing strand. Single molecule sequencing uses sensitive detection equipment and in some embodiments uses specialized solid phase substrates. Substrates for sequencing a single molecule typically include microfluidic devices or specialized wells such as zero wavelength guides capable of limiting the detection field to a small volume surrounding a single molecule. Detecting fluorescent emissions from a single molecule has been described for example in Single Molecule Detection and DNA Sequencing by Synthesis, Ph.D. Thesis of Emil P. Kartalov, California Institute of Technology (2004), and in U.S. Pat. Application No. 2003/0092005 by Levene et al, No. 2003/0044781 by Korlach et al, and No. 2003/0174992 by Levene et al, each of which is incorporated herein by reference.

In the Levene and Korlach references cited above, single molecule sequencing is described in terms of simultaneously using all four nucleoside triphosphates each labeled with an independently detectable label. These references, as with Kartalov, rely on detecting incorporation events by distinguishing a label incorporated into the growing nucleic acid chain. To detect a new incorporation event relative to an old incorporation event, each of these reference describe bleaching or quenching of the first incorporated label. Furthermore, Levene and Korlach describe using kinetic differences between non-specific nucleotide binding to the polymerase and productive binding where the nucleoside monophosphate is incorporated to determine an incorporation event. Because polymerases can incorporate hundreds to tens of thousands of bases per second, the ability to accurately detect each base incorporated presents a daunting detection and computational task. In contrast, the present methods are advantageous for use in single molecule sequencing because the rate of the polymerase elongation reaction is controlled by the rate of addition of the nucleotides. Moreover, because substrate degrading enzymes are used in various embodiments provided herein, bleaching or quenching is not required.

In various embodiments, the polyphosphate transfer enzyme may be added to the reaction mixture in the sample prior to, simultaneously with or after the polymerase has released the labeled polyphosphate. This allows the sequencing procedure to proceed without washing the template between successive nucleotide additions. Since washing steps are avoided in certain embodiments, it is not necessary to add new enzymes i.e. polymerase with each new nucleotide addition, thus improving the economy of the procedure. Thus, the nucleotide-degrading enzyme or enzymes are simply included in the polymerase reaction mix, and a sufficient time is allowed between each successive nucleotide addition for degradation of substantially most of the unincorporated nucleoside polyphosphates. The amount of substrate degrading enzymes to be used, and the length of time between nucleotide additions may readily be determined for each particular system, depending on the reactants selected, reaction conditions etc.

Alternatively, the substrate degrading enzyme(s) may be immobilized on a solid support i.e. a particulate solid support (i.e. magnetic beads) or a filter, or dipstick etc. and it may be added to the polymerase reaction mixture at a convenient time. For example such immobilized enzyme(s) may be added after nucleotide incorporation (i.e. chain extension) has taken place, and then, when the substrates are degraded the immobilized enzyme may be removed from the reaction mixture (i.e. it may be withdrawn or captured, i.e. magnetically in the case of magnetic beads), before the next nucleoside polyphosphate is added. The procedure may then be repeated to sequence more bases. Such an arrangement has the advantage that more efficient substrate degradation may be achieved as it permits more substrate degrading enzyme(s) to be added for a shorter period. This arrangement may also facilitate optimization of the balance between the two competing reactions of DNA polymerization and degradation of the nucleoside substrates.

Thus, in certain embodiments, the methods can be performed in sequential steps where at least one of the sample nucleic acid or the polymerase is immobilized on a surface substrate in a first reaction mixture forming a bound polymerase/sample nucleic acid complex. A first nucleoside polyphosphate (or nucleoside polyphosphate labeled at the terminal phosphate group with the donor dye) is added to the bound complex and the reaction is incubated for a sufficient period of time to allow the polymerase to incorporate the nucleotide into a growing polynucleotide chain with release of the polyphosphate (or polyphosphate labeled with the donor dye) into free solution. In a subsequent step, the solution is removed and transferred to a second reaction mixture containing the appropriate polyphosphate acceptor substrate and polyphosphate transfer enzyme. The second solution is monitored to detect whether fluorescence of the dually labeled polyphosphate acceptor substrate occurs, or whether the dye released from the quenched polyphosphate acceptor substrate is formed. If fluorescence is detected, then the identity of the nucleotide that is incorporated is determined.

For sequencing the sample nucleic acid, the process is repeated over several cycles, with a different nucleoside polyphosphate being added in subsequent cycles. Typically, the bound complex of polymerase and sample nucleic acid is washed with a suitable washing buffer between sequential additions of nucleoside polyphosphates to remove excess unbound substrates. In certain other embodiments, however, the substrate degrading enzyme or combination thereof is included in the second solution. In this case, a common second solution can be used for several cycles of addition because the fluorescence from any prior cycle is eliminated by the degrading activity of the appropriate substrate degrading enzymes.

In certain embodiments, after each cycle of addition and incubation with the nucleoside polyphosphate (or nucleoside polyphosphate labeled at the terminal phosphate group with the donor dye), the solution is transferred to a fresh second reaction mixture containing only the polyphosphate transfer enzyme and appropriate polyphosphate acceptor substrate. The second solution is monitored to determine whether fluorescence is emitted by the action of the polyphosphate transfer enzyme transferring the polyphosphate (or nucleoside polyphosphate labeled at the terminal phosphate group with the donor dye) to the appropriate acceptor substrate. After a sufficient period of time for the reaction to occur, the second solution is discarded whether or not fluorescence is detected. In these embodiments, it is not necessary to include a substrate degrading enzyme in the second reaction mixture.

Alternatively, instead of transferring the first solution to the second solution, to detect the fluorescence, a solution containing the polyphosphate transfer enzyme and the acceptor substrate may be added directly to the first solution after a sufficient time for the polymerase to release a polyphosphate. Fluorescence would then be detected in a common reaction vessel. The entire unbound components in the reaction vessel can then be removed and the bound complex washed to remove excess substrates. A second cycle is commenced by first adding a different nucleoside polyphosphate and incubating with the polymerase/sample nucleic acid complex, followed again by adding the second solution with the polyphosphate transfer enzyme and acceptor substrate to the reaction vessel to determine whether fluorescence is detected.

In other embodiments, the methods can be used for sequencing reactions that are continuously monitored in real time in a single reaction mixture. This is achieved by performing the polymerase chain extension reaction with sequential additions of different nucleoside polyphosphates at different time points in the presence of the appropriate polyphosphate transfer enzyme, polyphosphate acceptor substrate and substrate degrading enzymes. In these embodiments, the substrate degrading enzymes include an enzyme that also degrades each nucleoside polyphosphate that was added to the reaction mixture in a previous cycle. As mentioned above, apyrase is suitable for both degrading the polyphosphate acceptor substrate and the nucleoside polyphosphate. In embodiments using APS as the acceptor substrate that becomes detectable upon transfer of the polyphosphate labeled with the donor dye to APS labeled with the acceptor dye, the activity of apyrase causes the detected fluorescence to be reduced by removing the donor dye from proximity to the acceptor dye. In embodiments using APS dually labeled with the donor and quenching dye where the donor dye is released upon transfer of the polyphosphate, the activity of aryl sulfatase removes the sulfate from the released donor dye and thereby reduces the detected fluorescence. Thus, in either case, fluorescence is emitted for a brief period of time followed by a reduction in fluorescence, thereby forming a distinct signal indicative of the release of pyrophosphate by the enzymatic action of the polymerase.

As mentioned above, these embodiments permit polyphosphate release to be detected during the polymerase reaction giving a real-time signal. The rate limiting step in the series of reactions is the transfer of the released polyphosphate or labeled polyphosphate to the polyphosphate acceptor substrate. In both embodiments using APS as the polyphosphate acceptor substrate and ATP sulfurylase as the polyphosphate transfer enzyme, at least one non-natural substrate is being used. In the case of the donor/quencher embodiment, the non-natural substrate is APS labeled on the terminal sulfate group with the fluorescent dye and labeled on the base with the quenching dye. In the case of the donor/acceptor embodiment, the labeled polyphosphate released from the nucleoside polyphosphates and the APS labeled with the quenching dye are both non-natural substrates for the ATP sulfurylase. It has been demonstrated with other systems, however, that enzymes that use nucleotides as substrates are capable of recognizing modified nucleosides labeled with a dye on the base or the terminal phosphate group. For example, as mentioned earlier, nucleoside polyphosphates labeled on the terminal phosphate can be utilized as substrates by nucleic acid polymerases. It is also well known that nucleoside triphosphates labeled on the base with a fluorescent dye can also be utilized by polymerases. Hence, it is believed that the labeled APS, and the labeled pyrophosphate released by the enzymatic action of the polymerase can also be used as substrates by ATP sulfurylase. It is therefore likely that efficient transfer of the released polyphosphate, or polyphosphate labeled with the donor dye can be achieved in time frames from seconds to minutes.

In certain embodiments, the methods provided herein are useful for identifying the occurrence of single targeted nucleotide in the sample nucleic acid. These embodiments are useful for “genotyping” by determining single nucleotide polymorphisms present at a selected position in the sample nucleic acid. In such cases, a single stranded sample nucleic acid is combined with a primer that hybridizes immediately adjacent to the selected position. The identity of the nucleotide at the selected position is determined by identifying which nucleoside polyphosphate is incorporated by the DNA polymerase using the methods described herein. The identity of the nucleotide at a given position can be determined either by using separate reaction mixtures and adding a different nucleoside polyphosphates to each mixture to determining which mixture emits the fluorescence, or by sequentially adding different nucleoside polyphosphates to the same reaction mixture at different times until fluorescence is detected.

The methods provided herein based on fluorescent detection of polyphosphate released by the enzymatic action of a polymerase differ from conventional chemiluminescent detection of pyrophosphate, such as described in U.S. Pat. No. 6,258,568 to Nyren. One difference is that fluorescence is generally easier to monitor than chemiluminescent. Another difference is that fluorescence is more linear with substrate amount than chemiluminescence, which allows more accurate sequencing of nucleic acids having nucleotide repeats. Another difference is that there is no interference with a subsequent chemiluminescent reaction that uses luciferase when dATP, or ATP used in the sequencing reaction. Therefore, there is no need to substitute dATP, ddATP, or ATP with an analogue that is capable of acting as a substrate for a polymerase but incapable of acting as a substrate for the luciferase. However, if a user prefers to use such analogues, the methods provided herein are also capable of being implemented with those analogues without interfering with detracting from the operability of the methods. Thus, the methods may be used with any nucleoside analogue that can be used as a substrate by the polymerase, including but not limited to ATP [1-thio] triphosphate (or α-thiotriphosphate), deoxyadenosine 1-thioltriphospate, or deoxyadenosine α-thiotriphosphate (dATP αS) along with the α-thio analogues of CTP, GTP and TTP and deoxy and dideoxy versions of the same.

As mentioned herein before, the sample nucleic acid (i.e. DNA or RNA template) may conveniently be single-stranded, and may either by immobilized on a solid support or in solution in combination with a suitable primer. The use of the substrate degrading enzymes means that it is not necessary to immobilize the template DNA to facilitate washing, since a washing step is not required in various embodiments. By using thermostable enzymes, double-stranded DNA templates can also be used. In addition, the methods are suitable for sequencing using RNA polymerase and a doubled stranded template using RNA polymerase and terminally labeled nucleoside polyphospates. For example, sequencing with RNA polymerase can use a double stranded targeted DNA that is either modified to contain a promoter and RNA polymerase transcriptional initiation site, or that uses end initiation. The methods and compositions discussed in co-pending U.S. Application Nos. US 2003/0194740A1 and US 2001/0018184A1, as well as U.S. Pat. No. 6,306,607, incorporated herein by reference, for using terminal phosphate labeled nucleoside polyphosphates are particularly suitable for use in the methods provided herein that form a dually labeled donor/acceptor dye pair on the polyphosphate acceptor substrate. The methods provided herein using the donor/quencher pair on the polyphosphate acceptor substrate are also suitable for use with RNA polymerase and any nucleoside triphosphate or nucleoside polyphosphates substrate.

In still other embodiments, the present teachings are directed to kits including one or more of compounds, enzymes, dyes, or other components disclosed herein in any combination. The kits can optionally include instructions for using the components of the kits.

In still other embodiments, automated sequencing methods can be adapted to use the methods disclosed herein. Exemplary automated sequencing methods include use of instruments such as the Biotage PSQ™ HS 96 or the 455 Life Science Instrument System.

While the present teachings are described in conjunction with various embodiments, it is not intended that the present teaching be limited to such embodiments. On the contrary, the present teachings encompass various alternatives, modifications, and equivalents, as will be appreciated by those of skill in the art.

What is claimed is:

1. A method of analyzing a target nucleotide in a sample nucleic acid comprising
reacting the sample nucleic acid comprising the targeted nucleotide with a nucleotide polymerase, a nucleic acid primer which hybridizes to the sample nucleic acid, and a nucleoside polyphosphate molecule that is complementary to the targeted nucleotide and labeled with a first dye, for a time sufficient to release a first dye labeled polyphosphate molecule from the first-dye labeled nucleoside phosphate molecule;
reacting the first dye-labeled polyphosphate molecule with a polyphosphate acceptor substrate labeled with a second dye in the presence of polyphosphate transfer enzyme to form a polyphosphate acceptor substrate labeled with both the first dye and second dye, wherein one of the first dye and second dye is a fluorescence donor dye and the other dye is a fluorescence acceptor dye corresponding to the donor dye; and
applying excitation radiation within the absorption spectrum of the donor dye to produce emission radiation by the acceptor dye, said emission radiation identifying the targeted nucleotide.

2. The method of claim 1, wherein the polyphosphate molecule is pyrophosphate.

3. The method of claim 1 wherein the polyphosphate transfer enzyme is ATP sulfurylase.

4. The method of claim 1 wherein the polyphosphate acceptor substrate is adenosine 5′ phosphosulfate.

5. The method of claim 1 further including contacting the sample with a substrate degrading enzyme.

6. The method of claim 5 wherein the substrate degrading enzyme comprises apyrase.

7. The method of claim 5, further comprising applying heat to denature the substrate degrading enzyme.

8. The method of claim 1 wherein the donor dye is selected from the group consisting of fluorescein, rhodamine, and xanthene.

9. The method of claim 8, wherein the donor dye is fluorescein.

10. The method of claim 1 wherein the acceptor dye is selected from the group consisting of DABCYL, Symjaz 660, rhodamine, fluorescein, cyanine, pthalocyanine, and squaraine.

11. The method of claim 10, wherein the acceptor dye is DABCYL or Symjaz 660.

12. The method of claim 1, wherein the first dye is the donor dye and the second dye is the acceptor dye.

13. The method of claim 1, wherein the first dye is the acceptor dye and the second dye is the donor dye.

14. The method of claim 1, wherein the first dye is covalently bonded to the polyphosphate by a linker molecule.

15. The method of claim 1, wherein the second dye is covalently bonded to the polyphosphate substrate.

16. The method of claim 1 wherein the sample nucleic acid and polymerase are in a bound complex and at least one of the sample nucleic acid and the polymerase is immobilized on a substrate.

17. The method of claim 16 wherein the polyphosphate transfer enzyme and polyphosphate acceptor substrate are added to the sample after the bound complex is contacted with the nucleoside polyphosphate.

18. The method of claim 16 wherein the bound complex is contacted with the nucleoside polyphosphate in a first solution, and wherein at least a portion of the first solution is transferred to a second solution containing the polyphosphate transfer enzyme and polyphosphate acceptor substrate.

19. The method of claim 18 wherein the sample nucleotide and polymerase immobilized on the substrate are washed between the acts of contacting the sample with different first dye-labeled nucleoside polyphosphate.

20. The method of claim 1 further comprising providing nucleotide degrading enzyme present first dye-labeled nucleoside polyphosphate.

21. A method of identifying a targeted nucleotide comprising performing the method of claim 6 for one or more nucleotide triphosphate selected from the group consisting of dATP, dTTP, dGTP, and dCTP until fluorescence is detected at least once.

22. A method of sequencing a sample nucleic acid comprising:
identifying a first targeted nucleotide according to the method of claim 21; and
identifying a second targeted nucleotide according to the method of claim 21, wherein said second targeted nucleotide is 5′ of and adjacent to the first targeted nucleotide.

23. A method for detecting the presence of a polyphosphate in a sample, comprising contacting the sample with a polyphosphate transfer enzyme and a polyphosphate acceptor substrate labeled with a fluorescent dye that is quenched when linked to the acceptor substrate; and
detecting fluorescence from the fluorescent dye when the polyphosphate is transferred to the acceptor substrate and the fluorescent dye is released from the acceptor substrate by enzymatic action of the polyphosphate transfer enzyme.

24. The method of claim 23 wherein the polyphosphate acceptor substrate is labeled at a site other than the acceptor site with a quenching dye that quenches the fluorescence of the fluorescent dye.

25. The method of claim 23 wherein the polyphosphate is pyrophosphate.

26. The method of claim 23 further including contacting the sample with a substrate degrading enzyme that degrades the released fluorescent dye.

27. The method of claim 23 wherein the acceptor substrate is adenosine phosphosulfate (APS) and the polyphosphate transfer enzyme is ATP sulfurylase.

28. The method of claim 27 wherein the fluorescent dye is linked to a sulfate group of the APS and wherein the fluorescent dye is released with the sulfate and has relatively greater fluorescence than when the released fluorescent dye is not linked to the sulfate.

29. The method of claim 28 further including contacting the released fluorescent dye with a sulfatase that decreases the fluorescence of the released fluorescent dye.

30. The method of claim 27 wherein the fluorescent dye is linked to a sulfate group of the APS and wherein the fluorescent dye is released with the sulfate and has relatively lower fluorescence than when the released fluorescent dye is not linked to the sulfate.

31. The method of claim 30 further including contacting the released fluorescent dye with a sulfatase to release the fluorescent dye from the sulfate.

32. A method of analyzing a targeted nucleotide, comprising
reacting a sample nucleic acid comprising the targeted nucleotide with nucleic acid polymerase, a nucleic acid primer which hybridizes to the sample nucleic acid, and a nucleoside polyphosphate molecule for a time sufficient to release a polyphosphate molecule from the nucleoside polyphosphate molecule,
reacting the polyphosphate molecule with a polyphosphate transfer enzyme and a polyphosphate acceptor substrate labeled with a fluorescent dye at an acceptor site where the fluorescent dye is quenched when linked to the acceptor substrate; and
detecting fluorescence emitted from the fluorescent dye when a polyphosphate released from the nucleoside polyphosphate is transferred to the acceptor site on the acceptor substrate, thereby releasing the fluorescent dye from the acceptor substrate by enzymatic action of the polyphosphate transfer enzyme.

33. A method of identifying a targeted nucleotide comprising:
analyzing the targeted nucleotide according to the method of claim 32 for each of nucleotide polyphosphate selected from the group consisting of dATP, dTTP, dGTP, and dCTP until fluorescence is detected at least once.

34. The method of claim 32 wherein the polyphosphate acceptor substrate is labeled at a site other than the acceptor site with a quenching dye that quenches fluorescence of the fluorescent dye.

35. The method of claim 32 wherein the polyphosphate is pyrophosphate.

36. The method of claim 32 further comprising contacting the sample with a fluorescence degrading enzyme that degrades the released fluorescent dye.

37. The method of claim 32 wherein the acceptor substrate is APS and the polyphosphate transfer enzyme is ATP sulfurylase.

38. The method of claim 37 wherein the fluorescent dye is linked to a sulfate group of the APS and wherein the fluorescent dye is released with the sulfate and has relatively greater fluorescence than when the released fluorescent dye is not linked to the sulfate.

39. The method of claim 32 further including contacting the released fluorescent dye with a sulfatase that degrades the fluorescence of the released fluorescent dye.

40. The method of claim 32 wherein the fluorescent dye is linked to a sulfate group of the APS and wherein the fluorescent dye is released with the sulfate and has relatively lower fluorescence than when the released fluorescent dye is not linked to the sulfate.

41. The method of claim 32 further including contacting the released fluorescent dye with a sulfatase to release the fluorescent dye from the sulfate.

42. The method of claim 32 further including sequentially contacting the targeted nucleotide with different nucleoside until fluorescence is detected to thereby determine the identity of a nucleotide complementary to a nucleotide in the targeted nucleotide.

43. A method of sequencing a sample nucleic acid comprising:
identifying a first targeted nucleotide according to the method of claim 33; and
identifying a second targeted nucleotide which is 5′ of and adjacent to the first targeted nucleotide according to the method of claim 33.

44. The method of claim 43 wherein the sample further comprises a substrate degrading enzyme.

45. The method of claim 44 wherein the substrate degrading enzyme comprises at least one of apyrase and sulfatase.
